(19) United States
(12) Patent Application Publication
(10) Pub. No.: US 2023/0125425 A1
(43) Pub. Date: Apr. 27, 2023

(54) TRADITIONAL CHINESE MEDICINE EXTRACT COMPOSITION WITH FUNCTION OF REGULATING DEPRESSIVE EMOTION AND PREPARATION METHOD AND TRADITIONAL CHINESE MEDICINE PREPARATION THEREOF

(71) Applicant: CHENLAND NUTRITIONALS, INC., Pomona, CA (US)

(72) Inventors: Shengcan ZOU, Qingdao, Shandong (CN); Zengliang ZHANG, Qingdao, Shandong (CN); Wenyu LI, Qingdao, Shandong (CN); Shanglong WANG, Qingdao, Shandong (CN); Lei ZONG, Qingdao, Shandong (CN); Jiancheng ZONG, Qingdao, Shandong (CN)

(73) Assignee: CHENLAND NUTRITIONALS, INC., Pomona, CA (US)

(21) Appl. No.: 17/043,737

(22) PCT Filed: Dec. 20, 2019
(86) PCT No.: PCT/CN2019/126929

§ 371 Date: Sep. 30, 2020

(30) Foreign Application Priority Data
Nov. 14, 2019 (CN) 201911112026.4

(51) Int. Cl. A61K 36/38; A61K 36/65; A61P 25/24; A61K 36/254; A61K 9/00; A61K 9/20

(52) U.S. Cl. CPC A61K 36/38; A61K 36/65; A61P 25/24; A61K 36/254; A61K 9/0056; A61K 9/20; A61K 2236/333; A61K 2236/51

(57) ABSTRACT

The present disclosure relates to the technical field of traditional Chinese medicine, in particular to a traditional Chinese medicine extract composition with function of regulating depressive emotion and preparation method and traditional Chinese medicine preparation thereof. The composition consists of the following traditional Chinese medicine extracts in parts by weight: 40-80 parts of Hypericum perforatum extract, 10-40 parts of Acanthopanax senticosus extract, and 2-20 parts of Tree peony root bark extract. Compared with the prior art, the traditional Chinese medicine extract composition of the present disclosure has remarkable effect of alleviating depressive emotion, and is a safe and effective dietary supplement. In addition, it also has the effect of helping sleep. The composition has small side effects and no dependence, has a small dosage, and can be made into various dosage forms suitable for wide application.

### CROSS REFERENCE TO RELATED APPLICATIONS

This application claims the priority of Chinese Patent Application No. 201911112026.4 filed to the Chinese Patent Office on Nov. 14, 2019, titled “TRADITIONAL CHINESE MEDICINE EXTRACT COMPOSITION WITH FUNCTION OF REGULATING DEPRESSIVE EMOTION AND PREPARATION METHOD AND TRADITIONAL CHINESE MEDICINE PREPARATION THEREOF”, the entire contents of which are incorporated herein by reference.

### FIELD

The present disclosure relates to the field of traditional Chinese medicine, and in particular to a traditional Chinese medicine extract composition with function of regulating depressive emotion and preparation method and traditional Chinese medicine preparation thereof.

### BACKGROUND

Depression emotion is a common mood disorder disease, which seriously endangers people's physical and mental health. With the development of society, the pace of life has accelerated, competition for employment has intensified, and the number of people experiencing depression emotion has increased year by year. Severe depression emotion will develop into depression, people suffered from which are very suicidal, with a suicide rate of about 15%. In addition, about more than 90% of patients with mood disorders clinically can have sleep disorders in a certain period of time. Therefore, effective prevention and treatment of sleep disorders is of great significance for the relief and improvement of depression emotion.

Hypericum perforatum is bitter, astringent, and neutral in nature, and the action thereof is localized to the liver meridian. When used as a medicine, Hypericum perforatum has the effect of astringency and hemostasis, regulating menstruation and promoting lactation, clearing away heat and toxic materials, and removing dampness; and it mainly aims at hemoptysis, hematemesis, intestinal wind bleeding, metrorrhagia and metrostaxis, traumatic bleeding, irregular menstruation, breast milk stoppage for lactating women, jaundice, sore throat, swelling and pain of eye, urinary tract infection, mouth and nose sores, carbuncle furuncle swelling poison, and burn and scald. In the early 19th century, researchers discovered that Hypericum perforatum can be used to treat mood disorders. In the past two centuries, researches on the treatment of mental diseases of Hypericum perforatum are still ongoing.

Acanthopanax senticosus is pungent, slightly bitter, and warm in nature, and the action thereof is localized to the spleen, kidney and heart meridian. It has the effect of benefiting qi to invigorate the spleen and invigorating kidney for tranquillization and can be used for treating yang deficiency of spleen and kidney, physical weakness, loss of appetite, waist and knee pain, and insomnia and dreaminess. Acanthopanax senticosus has a long history of being widely used as a medicine in traditional Chinese medicine and has the function of “invigorating spleen-stomach and replenishing essence, strengthening the muscles and bones, and strengthening the mental power”. Acanthopanax senticosus can “lighten body and endure aging” after taking it for a long time, and the combination with other medicines can also “promote the diet, invigorate the strength, and not forgetful”. In addition, clinical studies have confirmed that Acanthopanax senticosus, like the traditional antidepressants imipramine, fluoxetine, etc., can help improve the mood and emotion of patients with depression, with fewer side effects and is safer and more effective.

Tree peony root bark is bitter, pungentin and slightly cold, and has the effects of clearing heat and cooling blood, promoting blood circulation to remove blood stasis. Studies have found that intraperitoneal injection or oral administration of paeonol in mice has sedative, hypnotic, and analgesic effects. Furthermore, paeonol can reduces the body temperature of normal mice (intraperitoneal injection or gavage) and also has an antipyretic effect on mice with artificial fever (caused by the injection of Bacillus typhi and Bacillus paratyphosus); and it is also resistant to shock induced by electric shock or drugs.

Currently, among the drugs used to treat depression, chemical drugs have obvious curative effects, but they have shortcomings such as narrow antidepressant spectrum, large adverse reactions, and easy recurrence. Therefore, people are turning to traditional Chinese medicine to fight depression. At present, Chinese Patent CN1381242A has disclosed a traditional Chinese medicine for treating depression, comprising Hypericum perforatum and Acanthopanax senticosus. The invention studied the antidepressant effect of the traditional Chinese medicine extract composition by mouse forced swimming test and tail suspension test and other indicators. Chinese Patent CN102670752A discloses the use of tree peony root bark extract in the preparation of a medicament for treating depression. However, the combination of Hypericum perforatum and Acanthopanax senticosus, and the tree peony root bark extract are not very effective for depression. No traditional Chinese medicine composition combing Hypericum perforatum, Acanthopanax senticosus and tree peony root bark has been disclosed.

### SUMMARY

In view of this, the present disclosure provides a traditional Chinese medicine extract composition with function of regulating depressive emotion and preparation method and traditional Chinese medicine preparation thereof. The composition has remarkable therapeutic effect on depression.

In order to achieve the above-mentioned purpose of the invention, the present disclosure provides the following technical solutions:

The present disclosure provides a traditional Chinese medicine extract composition with function of regulating depressive emotion, consisting of the following traditional Chinese medicine extracts in parts by weight: 40 to 80 parts of Hypericum perforatum extract, 10 to 40 parts of Acanthopanax senticosus extract, and 2 to 20 parts of tree peony root bark extract.

The present disclosure combines Hypericum perforatum extract, Acanthopanax senticosus extract and tree peony root bark extract in specific ratio to provide a composition having a significantly better effect on treating depression than the composition of Hypericum perforatum extract and Acanthopanax senticosus extract, and the tree peony root bark extract.

Preferably, the composition consists of the following traditional Chinese medicine extracts in parts by weight: 50 to 80 parts of Hypericum perforatum extract, 10 to 35 parts of Acanthopanax senticosus extract, and 2 to 10 parts of tree peony root bark extract.

Preferably, the composition consists of the following traditional Chinese medicine extracts in parts by weight: 64 to 77 parts of Hypericum perforatum extract, 13.3 to 25 parts of Acanthopanax senticosus extract, and 2.7 to 10 parts of tree peony root bark extract.

More preferably, the composition consists of the following traditional Chinese medicine extracts in parts by weight: 77 parts of Hypericum perforatum extract, 20 parts of Acanthopanax senticosus extract, and 3 parts of tree peony root bark extract.

The present disclosure also provides a method of preparing the traditional Chinese medicine extract composition with function of regulating depressive emotion, comprising the following steps:

(1) adding 10 to 12 times of weight of 60 vt % to 80 vt % ethanol aqueous solution containing 0.05% to 2% sodium hydroxide to Hypericum perforatum, extracting 2 times under heating and refluxing, 1.5 to 2.0 hours each time, combining the two filtrates and filtering, concentrating the filtrate into an extractum with a relative density of 1.11 to 1.13 at 72° C., and performing spray-drying to obtain the Hypericum perforatum extract;

(2) adding 8 to 10 times of weight of 0 vt % to 80 vt % ethanol aqueous solution to pieces of Acanthopanax senticosus, extracting 3 times under heating and refluxing, 1.0 to 2.5 hours each time, combining the three filtrates and filtering, concentrating the filtrate into an extractum with a relative density of 1.11 to 1.13 at 72° C., and performing spray-drying to obtain the Acanthopanax senticosus extract;

(3) adding 10 to 14 times of weight of water to tree peony root bark, heating and recovering 8 to 10 times amount of distillates, refrigerating for 20 to 24 hours, allowing crystallization and filtrating, and performing low-temperature drying to obtain the tree peony root bark extract; and

(4) mixing the Hypericum perforatum extract, the Acanthopanax senticosus extract and the tree peony root bark extract according to a combination ratio.

Preferably, the content of hyperoside in the Hypericum perforatum extract is 0.1% to 3%; the content of eleutheroside in the Acanthopanax senticosus extract is 0.3% to 5%; and the content of paeonol in the tree peony root bark extract is 80% to 99%.

Preferably, the method comprises the following steps:

(1) adding 10 times of weight of 80 vt % ethanol aqueous solution containing 0.1% sodium hydroxide to Hypericum perforatum, extracting 2 times under heating and refluxing, 1.5 hours each time, combining the two filtrates and filtering, concentrating the filtrate into an extractum with a relative density of 1.12 at 72° C., and performing spray-drying to obtain the Hypericum perforatum extract;

(2) adding 10 times of weight of aqueous solution to pieces of Acanthopanax senticosus, extracting 3 times under heating and refluxing, 1.0 hour each time, combining the three filtrates and filtering, concentrating the filtrate into an extractum with a relative density of 1.12 at 72° C., and performing spray-drying to obtain the Acanthopanax senticosus extract;

(3) adding 14 times of weight of water to tree peony root bark, heating and recovering 9 times amount of distillates, refrigerating for 24 hours, allowing crystallization and filtrating, and performing low-temperature drying to obtain the tree peony root bark extract; and

(4) mixing the Hypericum perforatum extract, the Acanthopanax senticosus extract and the tree peony root bark extract according to a combination ratio.

The present disclosure also provides a traditional Chinese medicine preparation, comprising the traditional Chinese medicine extract composition with function of regulating depressive emotion mentioned above and a pharmaceutically acceptable excipient.

Preferably, the dosage form of the traditional Chinese medicine preparation is a capsule or a tablet.

Preferably, the content of the traditional Chinese medicine extract composition with function of regulating depressive emotion is not less than 70% by weight in the traditional Chinese medicine preparation.

Preferably, the content of the traditional Chinese medicine extract composition with function of regulating depressive emotion is not less than 80% by weight in the traditional Chinese medicine preparation.

The invention provides a traditional Chinese medicine extract composition with function of regulating depressive emotion and preparation method and traditional Chinese medicine preparation thereof. The composition consists of the following traditional Chinese medicine extracts in parts by weight: 40 to 80 parts of Hypericum perforatum extract, 10 to 40 parts of Acanthopanax senticosus extract, and 2 to 20 parts of tree peony root bark extract. The present disclosure achieves the following technical effects:

The traditional Chinese medicine extract composition of the present disclosure can obviously alleviate the “behavioral despair” state of mice, reduce the number of static suspension and shorten the immobility time during tail suspension. Compared with the prior art, the traditional Chinese medicine extract composition of the present disclosure has obvious effects of alleviating depressive emotion, and is a safe and effective dietary supplement.

The present disclosure combines Hypericum perforatum extract, Acanthopanax senticosus extract and tree peony root bark extract in a specific ratio to provide a composition having a significantly better effect on treating depression than the composition of Hypericum perforatum extract and Acanthopanax senticosus extract, and the tree peony root bark extract.

The composition of the present disclosure not only has antidepressant effect, but also has the effect of helping sleep and the like. The composition has small side effects and no dependence, has a small dosage, and can be made into various dosage forms suitable for wide application.

### BRIEF DESCRIPTION OF DRAWINGS

FIG. 1 shows the effect of the test drugs on the number of static suspension in the forced swimming test of mice (x̅+SE); Note: normal control group (Control), fluoxetine group (FXT), St. John's wort extract group (LQ), traditional Chinese medicine composition low-dose group (TCMC-L), traditional Chinese medicine composition medium-dose group (TCMC-M), traditional Chinese medicine composition high-dose group (TCMC-H); *p<0.05, **p<0.01 vs. control group;

FIG. 2 shows the effect of the test drugs on the immobility time during the tail suspension test of mice (x̅±SE); Note: normal control group (Control), fluoxetine group (FXT), St. John's wort extract group (LQ), traditional Chinese medicine composition low-dose group (TCMC-L), traditional Chinese medicine composition medium-dose group (TCMC-M), traditional Chinese medicine composition high-dose group (TCMC-H); *p<0.05, **p<0.01 vs. control group;

FIG. 3 shows the effect of the test drugs on the total movement distance in the open field test of mice (x̅±SE); Note: normal control group (Control), fluoxetine group (FXT), St. John's wort extract group (LQ), traditional Chinese medicine composition low-dose group (TCMC-L), traditional Chinese medicine composition medium-dose group (TCMC-M), traditional Chinese medicine composition high-dose group (TCMC-H); *p<0.05, **p<0.01 vs. control group;

FIG. 4 shows the effect of the test drugs on the movement distance in the central area in the open field test of mice (x̅±SE); Note: normal control group (Control), fluoxetine group (FXT), St. John's wort extract group (LQ), traditional Chinese medicine composition low-dose group (TCMC-L), traditional Chinese medicine composition medium-dose group (TCMC-M), traditional Chinese medicine composition high-dose group (TCMC-H); *p<0.05, **p<0.01 vs. control group;

FIG. 5 shows the effect of the test drugs on the number of standing uprights in the open field test of mice (x̅±SE); Note: normal control group (Control), fluoxetine group (FXT), St. John's wort extract group (LQ), traditional Chinese medicine composition low-dose group (TCMC-L), traditional Chinese medicine composition medium-dose group (TCMC-M), traditional Chinese medicine composition high-dose group (TCMC-H); *p<0.05, **p<0.01 vs. control group;

FIG. 6 shows the effect of the test drugs on the body mass of model rats; Note: ****p<0.0001 vs. control group;

FIG. 7 shows the effect of the test drugs on the sucrose preference coefficient of model rats; Note: ****p<0.0001 vs. control group; ##p<0.01, ###p<0.001 vs. model group;

FIGS. 8-11 show the effect of the test drugs on the open field behavior of model rats; Note: *p<0.05, **p<0.01, ***p<0.001 vs. control group; #p<0.05 vs. model group;

FIG. 12 shows the effect of the test drugs on the static suspension time of model rats; Note: *p<0.05 vs. control group; #p<0.05 vs. model group.

### DETAILED DESCRIPTION

The invention discloses a traditional Chinese medicine extract composition with function of regulating depressive emotion and preparation method and traditional Chinese medicine preparation thereof. Those skilled in the art can learn from the content of the present disclosure and appropriately improve the process parameters. It should be particularly pointed out that all similar replacements and modifications are obvious to those skilled in the art, and are all deemed to be included in the present disclosure. The method and application of the present disclosure have been described through the preferred examples. Those skilled in the art can make changes or appropriate modifications and combinations to the methods and applications described herein without departing from the content, spirit and scope of the present disclosure, to implement and apply the technology of the present disclosure.

An object of the present disclosure is to provide a traditional Chinese medicine extract composition that overcomes the shortcomings of certain chemically synthesized antidepressant drugs. The traditional Chinese medicine extract composition can significantly alleviate the “behavioral despair” state of mice, reduce the number of static suspension and shorten the immobility time during tail suspension.

Another object of the present disclosure is to use the traditional Chinese medicine extract composition as dietary supplements to produce branded raw materials with stable properties and good effects for relieving depressive emotion and helping sleep.

The Hypericum perforatum extract according to the present disclosure should meet the effective substance content standard of Hypericum perforatum in the Chinese Pharmacopoeia 2015; and the Acanthopanax senticosus extract should meet the relevant standards of Hypericum perforatum in the Chinese Pharmacopoeia 2015.

The traditional Chinese medicine composition of the present disclosure is in dry powder form, can been produced by simple production process and is suitable for mechanized production, and can be made into various dosage forms suitable for wide application.

To achieve the purpose of the present disclosure, the present disclosure adopts the following technical solutions:

A traditional Chinese medicine extract composition with function of regulating depressive emotion, mainly consisting of the following raw medicines in weight percentage: 40% to 80% of Hypericum perforatum extract, 10% to 40% of Acanthopanax senticosus extract, and 2% to 20% of tree peony root bark extract.

The preferred weight percentage ranges of various raw medicines in the traditional Chinese medicine extract composition are: 65% to 80% of Hypericum perforatum extract, 10% to 35% of Acanthopanax senticosus extract, and 2% to 10% of tree peony root bark extract.

The optimal weight percentage of various raw medicines in the traditional Chinese medicine extract composition are: 77% of Hypericum perforatum extract, 20% of Acanthopanax senticosus extract, and 3% of tree peony root bark extract.

The method of preparing the traditional Chinese medicine extract composition of the present disclosure from the above-mentioned components comprises the following steps:

(1) providing the above-mentioned traditional Chinese medicine extracts by refluxing extraction method, comprising adding 10 to 12 times of amount of 60% to 80% ethanol containing 0.05% to 2% sodium hydroxide to Hypericum perforatum, extracting 2 times under heating and refluxing, 1.5 to 2.0 hours each time, combining the two filtrates and filtering, concentrating the filtrate into an extractum with a relative density of about 1.12, and performing spray-drying to obtain dried extractum powder A;

(2) adding 8 to 10 times amount of 0 to 80% ethanol to pieces of Acanthopanax senticosus, extracting 3 times under heating and refluxing, 1.0 to 2.5 hours each time, combining the filtrates and filtering, concentrating the filtrate into an extractum with a relative density of 1.12, and performing spray-drying to obtain dried extractum powder;

(3) adding 10 to 14 times of amount of water to tree peony root bark, heating and recovering 8 to 10 times amount of distillates, refrigerating for 20 to 24 hours, allowing crystallization and filtrating, and performing low-temperature drying;

(4) in the above three extracts, the content of hyperoside, the main ingredient of extract A, is 0.1% to 3%; the content of eleutheroside, the main ingredient of extract B, is 0.3% to 5%; and the content of paeonol, the main ingredient of extract C, is 80% to 99%; and

(5) mixing the above three extracts according to a weight ratio and adding appropriate amount of pregelatinized starch, talcum power, magnesium stearate, etc, and filling the mixture into capsules or press the mixture into tablets to obtain the traditional Chinese medicine composition.

The raw materials or auxiliary materials used in the traditional Chinese medicine extract composition with function of regulating depressive emotion and the preparation method and traditional Chinese medicine preparation thereof provided by the present disclosure can all be purchased from the market.

The following examples further illustrate the present disclosure.

### Example 1

This example provides a method for preparing a traditional Chinese medicine composition with function of regulating depressive emotion. The traditional Chinese medicine composition of the present disclosure for improving depressive emotion and sleep consists of Hypericum perforatum extract, Acanthopanax senticosus extract and tree peony root bark extract. The composition was prepared by a method comprising the following steps:

adding 10 times amount of 70% ethanol to Hypericum perforatum, extracting 2 times under heating and refluxing, 1.5 hours each time, combining the two filtrates and filtering, concentrating the filtrate into an extractum with a relative density of 1.12 at 72° C., and performing spray-drying to obtain a dried extractum powder as the Hypericum perforatum extract, in which the content of hyperoside was 1.43%;

adding 10 times amount of 80% ethanol to pieces of Acanthopanax senticosus, extracting 3 times under heating and refluxing, 1.0 hour each time, combining the three filtrates and filtering, concentrating the filtrate into an extractum with a relative density of 1.2 at 72° C., and performing spray-drying to obtain a dried extractum powder as the Acanthopanax senticosus extract, in which the content of eleutheroside was 1.06%;

adding 14 times amount of water to tree peony root bark, heating and recovering 9 times amount of distillates, refrigerating for 24 hours, allowing crystallization and filtrating, and performing low-temperature drying to obtain the tree peony root bark extract, in which the content of paeonol was 98%; and

mixing the Hypericum perforatum extract, the Acanthopanax senticosus extract and the tree peony root bark extract according to the weight ratio of 70%:25%:5%, to obtain the composition.

### Example 2

This example provides a method of preparing a traditional Chinese medicine composition with function of regulating depressive emotion, comprising the following steps:

adding 10 times amount of 80% ethanol to Hypericum perforatum, extracting 2 times under heating and refluxing, 1.5 hours each time, combining the two filtrates and filtering, concentrating the filtrate into an extractum with a relative density of 1.12 at 72° C., and performing spray-drying to obtain a dried extractum powder as the Hypericum perforatum extract, in which the content of hyperoside was 1.44%;

adding 10 times amount of 50% ethanol to pieces of Acanthopanax senticosus, extracting 3 times under heating and refluxing, 1.0 hour each time, combining the three filtrates and filtering, concentrating the filtrate into an extractum with a relative density of 1.2 at 72° C., and performing spray-drying to obtain a dried extractum powder as the Acanthopanax senticosus extract, in which the content of eleutheroside was 0.88%;

adding 14 times amount of water to tree peony root bark, heating and recovering 9 times amount of distillates, refrigerating for 24 hours, allowing crystallization and filtrating, and performing low-temperature drying to obtain the tree peony root bark extract, in which the content of paeonol was 98%; and

mixing the Hypericum perforatum extract, the Acanthopanax senticosus extract and the tree peony root bark extract according to the weight ratio of 70%:20%:10%, to obtain the composition.

### Example 3

This example provides a method for preparing a traditional Chinese medicine composition with function of regulating depressive emotion, comprising the following steps:

adding 10 times amount of 80% ethanol containing 0.1% sodium hydroxide to Hypericum perforatum, extracting 2 times under heating and refluxing, 1.5 hours each time, combining the two filtrates and filtering, concentrating the filtrate into an extractum with a relative density of 1.12 at 72° C., and performing spray-drying to obtain a dried extractum powder as the Hypericum perforatum extract, in which the content of hyperoside was 1.29%;

adding 10 times amount of aqueous solution to pieces of Acanthopanax senticosus, extracting 3 times under heating and refluxing, 1.0 hour each time, combining the three filtrates and filtering, concentrating the filtrate into an extractum with a relative density of 1.2 at 72° C., and performing spray-drying to obtain a dried extractum powder as the Acanthopanax senticosus extract, in which the content of eleutheroside was 0.64%;

adding 14 times amount of water to tree peony root bark, heating and recovering 9 times amount of distillates, refrigerating for 24 hours, allowing crystallization and filtrating, and performing low-temperature drying to obtain the tree peony root bark extract, in which the content of paeonol was 98%; and

mixing the Hypericum perforatum extract, the Acanthopanax senticosus extract and the tree peony root bark extract according to the weight ratio of 77%:20%:3%, to obtain the composition.

### Example 4 Preparation of Capsules

This example provides a method of preparing a traditional Chinese medicine composition with function of regulating depressive emotion, comprising the following steps:

(1) adding 10 times amount of 80% ethanol containing 0.1% sodium hydroxide to Hypericum perforatum, extracting 2 times under heating and refluxing, 1.5 hours each time, combining the two filtrates and filtering, concentrating the filtrate into an extractum with a relative density of 1.12 at 72° C., and performing spray-drying to obtain the Hypericum perforatum extract;

(2) adding 10 times amount of aqueous solution to pieces of Acanthopanax senticosus, extracting 3 times under heating and refluxing, 1.0 hour each time, combining the three filtrates and filtering, concentrating the filtrate into an extractum with a relative density of 1.2 at 72° C., and performing spray-drying to obtain the Acanthopanax senticosus extract;

(3) adding 14 times amount of water to tree peony root bark, heating and recovering 9 times amount of distillates, refrigerating for 24 hours, allowing crystallization and filtrating, and performing low-temperature drying to obtain the tree peony root bark extract; and

(4) concentrating the extracts obtained above to a relative density of 1.08 to 1.10, performing spray-drying and then mixing evenly according to a weight ratio; passing the mixture powder through a 40-mesh sieve, then adding pregelatinized starch, talcum powder and magnesium stearate and mixing evenly to obtain a mixture comprising the Hypericum perforatum extract, the Acanthopanax senticosus extract, the tree peony root bark extract and auxiliary materials in a ratio of 64.0%:13.3%: 2.7%: 20.0%, and then filling the mixture in to capsules obtain the capsule preparation.

### Example 5 Preparation of Tables

This example provides a method for preparing a traditional Chinese medicine composition with function of regulating depressive emotion, comprising the following steps:

(1) adding 10 times amount of 80% ethanol containing 0.1% sodium hydroxide to Hypericum perforatum, extracting 2 times under heating and refluxing, 1.5 hours each time, combining the two filtrates and filtering, concentrating the filtrate into an extractum with a relative density of 1.12 at 72° C., and performing spray-drying to obtain the Hypericum perforatum extract;

(2) adding 10 times amount of aqueous solution to pieces of Acanthopanax senticosus, extracting 3 times under heating and refluxing, 1.0 hour each time, combining the three filtrates and filtering, concentrating the filtrate into an extractum with a relative density of 1.2 at 72° C., and performing spray-drying to obtain the Acanthopanax senticosus extract;

(3) adding 14 times amount of water to tree peony root bark, heating and recovering 9 times amount of distillates, refrigerating for 24 hours, allowing crystallization and filtrating, and performing low-temperature drying to obtain the tree peony root bark extract; and

(4) concentrating the extracts obtained above to a relative density of 1.08 to 1.10, performing spray-drying and then mixing according to a weight ratio, adding starch and magnesium stearate and mixing evenly to obtain a mixture comprising the Hypericum perforatum extract, the Acanthopanax senticosus extract, the tree peony root bark extract, starch and magnesium stearate in a ratio of 64.0%:13.3%:2.7%:15.0%:5.0%, and then pressing the mixture into tablets and coating thin film to obtain the tablet preparation.

### Example 6

The following test examples are used to further illustrate the beneficial effects of the traditional Chinese medicine composition of the present disclosure on anti-depressive emotions.

1 Materials and Methods

1.1 Materials

1.1.1 Instruments and consumables: SuperMaze animal behavior analysis system: Shanghai XinRuan Information Technology Co., Ltd.; SMART 3.0 Behavioral Video Analysis Software: DL Naturegene Life Sciences, Inc; open field test system: Shanghai XinRuan Information Technology Co., Ltd.; forced tail suspension test system: Shanghai XinRuan Information Technology Co., Ltd.; electronic balance: Sartorius, Germany, mouse intragastric administration device: Jinan Yiyan Technology Development Co., Ltd.

1.1.2 Drugs: test drug, numbered as MH-1 (composition of Example 3). Fluoxetine hydrochloride capsules: Eli Lilly Suzhou Pharmaceutical Co., Ltd., strength: 20 mg/capsule (7 capsules/box), batch number: 7704A. St. John's wort extract tablets: Dr. Willmar Schwabe GmbH & Co. KG, Germany, strength: 0.56 g/tablet (15 tablets/box), batch number: 0141217.

1.1.3 Animals: 108 SPF grade male C57BL/6J mice, body mass of 20±2 g, provided by Jinan Pengyue Experimental Animal Breeding Co., Ltd., license number: SCXK (Lu) 2018-0003.

1.2 Method

1.2.1 Feeding Conditions and Grouping

The mice were kept in a light, temperature and humidity controlled room with 6 mice per cage, and were allowed to eat and drink ad libitum: temperature 21±2° C. humidity 50±10%, 12 h/12 h light and dark cycle (light on at 20:00, light off at 8:00). All animal experiments were conducted in accordance with the guidelines on experiment animal welfare and use issued by NIH.

The mice were adapted for one week after their arrival, and then entered the experiment. The mice were divided into 6 groups according to the random block design method, with 12 mice in each group: normal control group (Control), fluoxetine group (FXT), St. John's wort extract group (LQ), traditional Chinese medicine composition low-dose group (TCMC-L), traditional Chinese medicine composition medium-dose group (TCMC-M), and traditional Chinese medicine composition high-dose group (TCMC-H).

1.2.2 Mode of Administration and Course of Treatment

The normal group was given the same volume of purified water by gavage, and the gavage doses of other groups are shown in the table in the Experimental results. The mice were administrated by gavage at a fixed time (9:00) every morning, with a gavage volume of 0.1 mL·10g⁻¹, for one month. The behavioral test was performed 1 h after the last gavage.

1.2.3 Experimental Method

1.2.3.1 Forced Swimming Test of Mice

During forced swimming, all animals were put into a plexiglass cylinder containing water with a deep of 20 cm at 23±1° C., individually. After 15 minutes, they were transferred to a dry environment at 30° C. for 30 minutes (pre-experiment). After 24 hours, the mice were put into the plexiglass cylinder again for 5 minutes (formal experiment). During this period, the mice were recorded with a video camera. After each mouse was tested, the water was changed and the cylinder was cleaned. SMART3.0 software was used to record the number of static suspension and the latency of static suspension.

1.2.3.2 Tail Suspension Test of Mice

In the tail suspension test, mice were hung on the tail suspension device using medical tape at a position ⅓ from the tip of the tail, in an upright position with the head being about 30 cm from the ground and facing straight onto a camera, which was used to record the immobility time during the tail suspension test of the mice in each group. The test lasted for 6 minutes, and the last 4 minutes were used to calculate the immobility time during the tail suspension test of mice. Immobility time is defined as the time when the mouse stops struggling and is still in a hanging state.

1.2.3.3 Open Field Test of Mice

The test was carried out in an open field box with a length of 50 cm, a width of 50 cm and a depth of 50 cm. The bottom surface of the open field box was equally divided into 9 squares, in which the middle area was the central area, and the remaining areas were the peripheral areas. The bottom and four sides of the open field box were all white. The test was carried out in a quiet and weak red light environment. At the beginning, the mice were placed in the center of the open field box to observe their activities within 6 minutes. The total movement distance, the movement distance in the central area and the number of standing upright of the mice were recorded.

2 Experimental Results

2.1 Forced Swimming Test

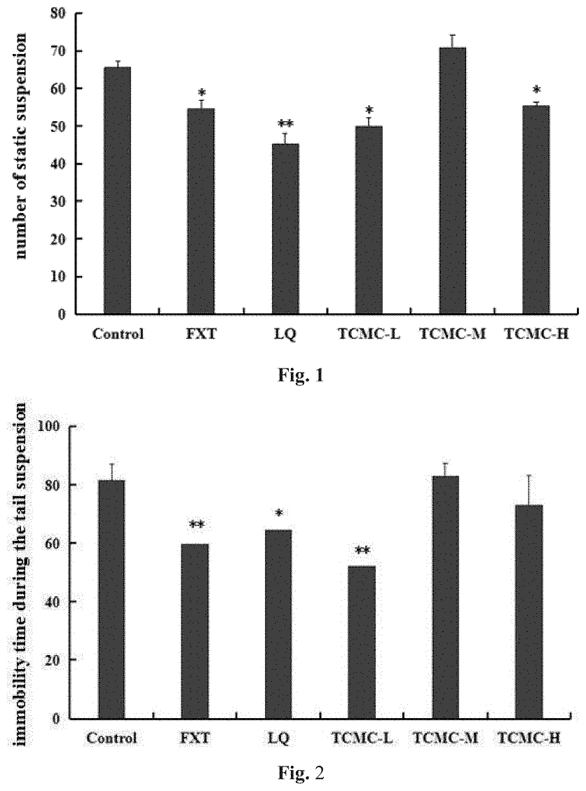

The forced swimming test of classical depression animal model was used to observe the effect of continuous administration by gavage of different doses of the traditional Chinese medicine extract composition of the present disclosure for reducing the immobility time (sec) during forced swimming of mice, to evaluate the different effective doses of the traditional Chinese medicine extract composition of the present disclosure. The experimental results shows that, after administration by gavage, the numbers of static suspension of mice in the fluoxetine group, the St. John's wort extract group, and the 84.625 mg/kg dose group and the 338.5 mg/kg dose group of the traditional Chinese medicine extract composition of the present disclosure were significantly less than that of the control group, suggesting that fluoxetine, the St. John's wort extract, and 84.625 mg/kg dose and 338.5 mg/kg dose of the traditional Chinese medicine extract composition of the present disclosure can significantly alleviate the “behavioral despair” state of mice and reduce the numbers of static suspension (Table 1, FIG. 1).

TABLE 1
The effect of the test drugs on the number of static suspension in
the forced swimming test of mice (x̅ ± SE)

| | | Number of | Number of static |
|---|---|---|---|
| Group | Dose (mg/kg) | animals | suspension |
| Control | 0 | 11 | 65.55 ± 4.611 |
| FXT | 3.33 | 11 | 54.64 ± 2.704* |
| LQ | 186.67 | 10 | 45.25 ± 4.695** |
| TCMC-L | 84.625 | 12 | 49.92 ± 5.126* |
| TCMC-M | 169.25 | 12 | 70.92 ± 4.435 |
| TCMC-H | 338.5 | 12 | 55.25 ± 3.778* |

Note:
*p < 0.05,
**p < 0.01 vs. Control Group

2.2 Tail Suspension Test

The tail suspension test of classical depression animal model was used to observe the effect of continuous administration by gavage of different doses of the traditional Chinese medicine extract composition of the present disclosure for reducing the immobility time (sec) during the tail suspension test of mice, to evaluate the different effective doses of the traditional Chinese medicine extract composition of the present disclosure. The experimental results shows that, after administration by gavage, the immobility time during the tail suspension test of mice in the fluoxetine group, the St. John's wort extract group, and the 84.625 mg/kg dose group were significantly less than that of the control group, suggesting that fluoxetine, the St. John's wort extract, and 84.625 mg/kg dose of the traditional Chinese medicine extract composition of the present disclosure can significantly alleviate the “behavioral despair” state of mice and reduce the immobility time during the tail suspension test of mice (Table 2, FIG. 2).

TABLE 2
The effect of test drugs on the immobility time during the
tail suspension test of mice (x̅ ± SE)

| | | Number of | Immobility time |
|---|---|---|---|
| Group | Dose (mg/kg) | animals | during tail suspension |
| Control | 0 | 11 | 81.37 ± 5.817 |
| FXT | 3.33 | 10 | 59.62 ± 6.270** |
| LQ | 186.67 | 9 | 64.53 ± 7.152* |
| TCMC-L | 84.625 | 10 | 52.21 ± 7.771** |
| TCMC-M | 169.25 | 10 | 82.68 ± 4.733 |
| TCMC-H | 338.5 | 11 | 73.02 ± 10.15 |

Note:
*p < 0.05,
**p < 0.01 vs. Control Group

2.3 Open Field Test

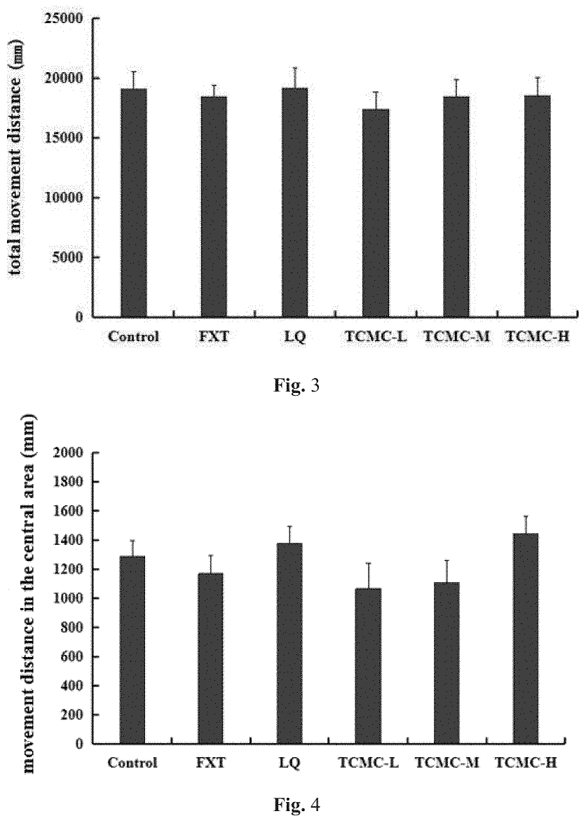

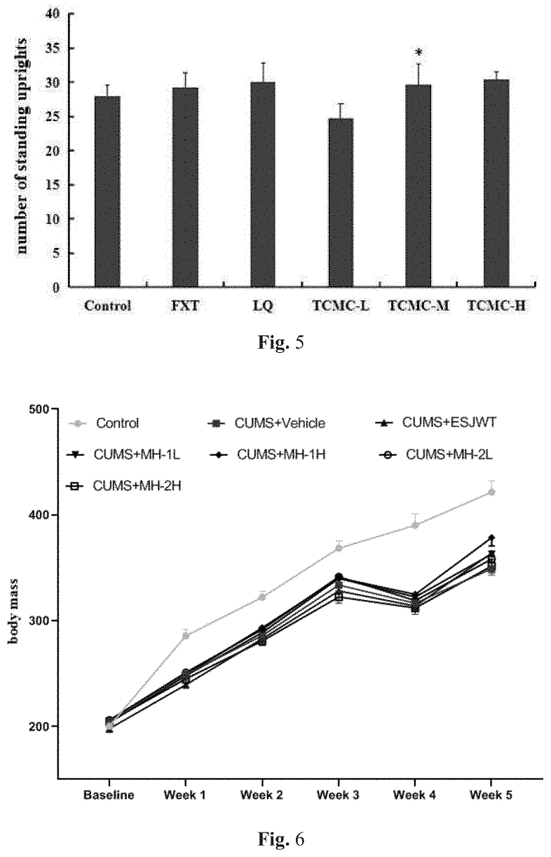

There were no significant change in the total movement distance, movement distance in the central area and the number of standing uprights of mice in the fluoxetine group, the St. John's wort extract group, and each dose group of the traditional Chinese medicine extract composition, compared with that of mice in the control group (P>0.05), see Table 3 to Table 5, FIG. 3 to FIG. 5.

In the forced swimming test and the tail suspension test, some drugs may have false positive or false negative results, since certain psychoactive drugs can reduce the immobility time of mice by enhancing the spontaneous activity of animals. In order to avoid false positive or false negative reactions, open field tests are often used to observe the spontaneous activity of animals. In this experiment, we observed the total movement distance and the movement distance in the central area of the animal, and the number of standing upright of animal in the 6 minutes of the open field test. The spontaneous activity of the mice after treatment with the traditional Chinese medicine extract composition of the present disclosure did not change significantly, indicating that the reduction in immobility time of mice in the forced swimming test and the tail suspension test is related to the antidepressant activity of the traditional Chinese medicine extract composition of the present disclosure, rather than the increase in spontaneous activity of mice caused by drug stimulation. The results of this experiment suggest that the traditional Chinese medicine extract composition of the present disclosure has an antidepressant effect.

TABLE 3
The effect of the test drugs on the total movement distance in
the open field test of mice (x̅ ± SE)

| | | Number of | Total movement |
|---|---|---|---|
| Group | Dose (mg/kg) | animals | distance |
| Control | 0 | 9 | 19083 ± 1395 |
| FXT | 3.33 | 10 | 18435 ± 950.6 |
| LQ | 186.67 | 10 | 19151 ± 1678 |
| TCMC-L | 84.625 | 10 | 17398 ± 1443 |
| TCMC-M | 169.25 | 11 | 18441 ± 1464 |
| TCMC-H | 338.5 | 10 | 18472 ± 1545 |

TABLE 4
The effect of the test drugs on the movement distance in the central
area in the open field test of mice (x̅ ± SE)

| | | | Movement distance |
|---|---|---|---|
| | | Number of | in the central area |
| Group | Dose (mg/kg) | animals | (mm) |
| Control | 0 | 9 | 1285 ± 112.2 |
| FXT | 3.33 | 10 | 1167 ± 126.6 |
| LQ | 186.67 | 10 | 1377 ± 117.3 |
| TCMC-L | 84.625 | 10 | 1066 ± 172.9 |
| TCMC-M | 169.25 | 11 | 1104 ± 156.1 |
| TCMC-H | 338.5 | 10 | 1441 ± 126.0 |

TABLE 5
The effect of the test drugs on the number of standing uprights
in the open field test of mice (x̅ ± SE)

| | | Number of | Number of |
|---|---|---|---|
| Group | Dose (mg/kg) | animals | standing uprights |
| Control | 0 | 9 | 27.89 ± 1.654 |
| FXT | 3.33 | 10 | 29.20 ± 2.133 |
| LQ | 186.67 | 10 | 30.00 ± 2.777 |
| TCMC-L | 84.625 | 10 | 24.60 ± 2.252 |
| TCMC-M | 169.25 | 11 | 29.55 ± 3.178 |
| TCMC-H | 338.5 | 10 | 30.40 ± 1.166 |

### Example 7

### (1) Acute Toxicity

Acute toxicity test of SD rats administrated by gavage: 5 male rats and 5 female rats were administrated with a solution of 0.3 g/mL drug, at low, medium and high doses of 4, 8, 12 g/kg respectively, which were approximately 300, 600, and 900 times of the clinical dose. The blank control group was given the same dose of pure water. Animal were administered twice by gavage, with an interval of 6 hours, and observed for 14 days. During the experiment, the animals had normal activity, normal weight gain, no adverse reactions, and the mortality of animal was 0%.

### (2) Long-Term Toxicity:

80 SD rats, half male and half male, were fed in separate cages. Those animal were randomly divided into four groups, each group with 20 animals: control group, low-dose group (0.3 g/kg, about 23 times the clinical dose), middle-dose group (0.9 g/kg, about 68 times the clinical dose), and high-dose group (3 g/kg, about 230 times the clinical dose). The animals were administered 7 days a week for 13 consecutive weeks. The appearance and physical signs, behavioral activities, glandular secretion, breathing, feces and the like of animals were observed daily. During the experiment, all indicators of the animal were normal, and the animal had no adverse reactions.

### Example 8

In the following test examples, the Chronic Unpredictable Mild Stress (CUMS) rat model was used to test different traditional Chinese medicines for the antidepressant effects.

1. Materials and Methods

1.1 Materials

1.1.1 Instruments and consumables: SMART 3.0 video tracking system: Harvard Apparatus; SuperMaze Animal behavior analysis system: Shanghai XinRuan Information Technology Co., Ltd.; open field test system: Shanghai XinRuan Information Technology Co., Ltd.; forced tail suspension test system: Shanghai XinRuan Information Technology Co., Ltd.; electronic balance: Sartorius, Germany; rat intragastric administration device: Jinan Yiyan Technology Development Co., Ltd; Speed Adjusting Multi-purpose oscillator: Guohua Electric Appliance Co., Ltd.

1.1.2 Drugs: test drugs, including MH-1 low dose, MH-1 high dose, MH-2 low dose, and MH-2 high dose. St. John's Wort extract tablets (Hypericum perforatum tablets): GmbH & Co. KG Germany, specification: 0.56 g/tablet (15 tablets/box), batch number: 0141217.

MH-1 low dose: a traditional Chinese medicine composition of Hypericum perforatum extract, Acanthopanax senticosus extract, tree peony root bark extract (77%, 20%, 3%, respectively), used at the same dose as the low-dose group in Example 6.

MH-1 high dose: a traditional Chinese medicine composition of Hypericum perforatum extract, Acanthopanax senticosus extract, tree peony root bark extract (77%, 20%, 3%, respectively), used at the same dose as the medium-dose group in Example 6.

MH-2 low dose: a traditional Chinese medicine composition consists of Hypericum perforatum extract and Acanthopanax senticosus extract at a ratio of 79.4%:20.6%, used at the same dose as the MH-1 high dose group.

MH-2 high dose: a traditional Chinese medicine composition consists of Hypericum perforatum extract and Acanthopanax senticosus extract at a ratio of 79.4%:20.6%, used at a dose twice that of the MH-1 high dose group.

1.1.3 Animals: 84 SPF grade male Wistar rats, body mass of 250-320 g, provided by Jinan Pengyue Experimental Animal Breeding Co., Ltd., license number: SCXK (Lu) 2018-0003.

1.2 Method

1.2.1 Feeding Conditions and Grouping

Rats were routinely housed in a standardized environment at room temperature of 22-24° C., a humidity of 50-60/a, and a 12h/12h light-dark cycle (light off at 8:00 in the morning and light on at 8:00 in the evening), and were allowed to eat and drink ad libitum. The experiment was carried out in strict accordance with the use regulations of laboratory animal. Before the experiment, the animals were adapted to the experimental environment for one week. Before the start of the experiment, the rats were detected for the baseline value of sucrose preference and the open field test, and were randomly grouped into 7 groups, each group with 12 animals: blank control group (Control), CUMS depression model group (CUMS+Vehicle), CUMS+Hypericum perforatum tablet treatment group (CUMS+ESJWT), CUMS+MH-1 low-dose treatment group (CUMS+MH-1L), CUMS+MH-1 high-dose treatment group (CUMS+MH-1H), CUMS+MH-2 low-dose treatment group (CUMS+MH-2L) and CUMS+MH-2 high-dose treatment group (CUMS+MH-2H), according to the sucrose preference value and the total movement distance in the open field of the rats. Rats in the blank control group were raised in population and allowed to eat and drink normally without any stimulation. The remaining groups were used to establish CUMS depression rat models through chronic unpredictable stress combined with individual feeding.

1.2.2 Method of Administration and Course of Treatment

At a fixed time (9:00) every day, the normal control group and the model group were given an equal volume of distilled water by gavage, and the treatment groups were given different test drugs. The administration volume was 0.1 mL·10 g⁻¹ and the administration continued throughout the whole process of stress. One hour after gavage administration, the behavioral test was performed. The next morning after the behavioral test was completed, the animals were sacrificed and the materials were collected and stored in a low-temperature refrigerator.

1.2.3 Establishment of Animal Model

The remaining 72 rats were subjected to two or three random, mild and unpredictable stresses every day, with no same stimuli in three consecutive days, including: day-night reversal (24 h), 45° inclined cage (12 h), fetter (30 min), wet cage (12 h), food deprivation (12 h), swimming in iced water at 18° C. (10 min), strobe lighting (12 h), white noise (15 min), shaking cage (15 min), and the like. All rats were feed individually, and subjected to chronic mild stimulation for 5 weeks. The sucrose preference of the rats was measured until the model was successfully established. During the modeling process, each rat was subjected to body mass measure, sucrose preference coefficient measure, and open field test once a week, until the end of the administration. A forced swimming test was performed before the final sacrifice.

1.2.4 Behavioral Evaluation

1.2.4.1 Sucrose Preference Test

The sucrose preference test was used to reflect the anhedonia symptoms in animals with depression. After the rats were deprived of water for 12 hours, each rat was given pre-weighed sterilized drinking water and 1% sucrose water. In order to prevent the rats from having a position preference, the positions of the two water bottles were exchanged after 12 hours. The rats were allowed to drink ad libitum, and the amount of drinking water and sucrose ingested by the rats within 24 hours were recorded. According to the calculation formula of the sucrose preference coefficient below, the sucrose preference coefficient of each rat was calculated.

$$SP = \frac{sucrose intake ( g )}{sucrose intake ( g ) + water intake ( g )} \times 100$$

1.2.4.2 Open Field Test

The open field test can be used to assess the physical activity, exploration interest and anxiety level of animals in an unfamiliar environment. The animal behavior analysis system was used to record and analyze the activities of rats in the open field test box. In a quiet environment, the rats were carefully placed in the center of the open field test box. The activities of the rats within 6 minutes were recorded by an infrared camera system and video synthesizer, and the movement trajectories of the rats were analyzed by a computer analysis system. The behavioral indicators recorded during the experiment included the total movement distance, the number of standing uprights, the movement distance in the central area and the stay time in the central area. The total movement distance reflects the activity of the rats' limbs, the number of standing uprights reflects the exploration interest of the rats, and the movement distance and the stay time in the central area reflect the anxiety level of rats. After the experiment of each rat, the residue in the open field box was cleaned to remove the smell of the previous rat, so that the performance of the next rat was not affected.

1.2.4.3 Forced Swimming Test

The forced swimming test is used to reflect the behavioral despair of depression animals, and can also be used to screen antidepressants. The rat was individually placed in a transparent cylinder (height 40 cm and diameter 20 cm) containing water with a depth of about 30 cm. The temperature was controlled at 22±1° C. All animals were placed in the water environment for 15 minutes, and then transferred to a dry environment at 30° C. for 30 minutes (preliminary experiment). After 24 hours, the rat was placed in the plexiglass cylinder again for 5 minutes (formal experiment). The SMART 3.0 video tracking system was used to record the static suspension time of the rat within 5 minutes, and the rat was dried with a towel immediately after the forced swimming.

1.2.5 Statistical Methods

The data was analyzed using GraphPad Prism 8.02 software (GraphPad Software, Inc., San Diego, Calif., USA). Data of all groups were subjected to normality test (Kolmogorov-Smirnov test) and homogeneity of variance test (Levene test) before parameter testing. One-way analysis of variance was used for the number of standing uprights and the static suspension time, two-way analysis of variance was used for other test parameters, and Bonferroni's multiple comparisons test was used for post-hoc test. The test level was set to p<0.05.

2. Experimental Results

### 2.1 Body Mass

The test results are shown in Table 6 and FIG. 6:

TABLE 6
The effect of the test drugs on the body mass of model rats (x ± SE)

| | Dose | Number of | | | | | | |
|---|---|---|---|---|---|---|---|---|
| Group | (mg/kg) | animals | Baseline | Week 1 | Week 2 | Week 3 | Week 4 | Week 5 |
| Control | — | 14 | 200.4 ± 2.79 | 285.7 ± 6.13 | 322.2 ± 5.69 | 368.6 ± 6.93 | 390.3 ± 10.61 | 421.6 ± 10.47 |
| CUMS + | — | 12 | 204.9 ± 2.97 | 249.4 ± 4.51 | 286.0 ± 5.00 | 333.8 ± 6.54 | 316.5 ± 6.50 | 349.2 ± 6.06**** |
| Vehicle | | | | | | | | |
| CUMS + | 117.60 | 12 | 198.0 ± 3.62 | 239.5 ± 3.22 | 283.0 ± 3.99 | 328.1 ± 6.00 | 313.9 ± 5.61 | 363.9 ± 5.83**** |
| ESJWT | | | | | | | | |
| CUMS + | 53.31 | 13 | 203.7 ± 3.02 | 248.0 ± 4.23 | 288.9 ± 4.92 | 339.9 ± 7.06 | 322.4 ± 6.64 | 363.0 ± 7.10**** |
| MH-1L | | | | | | | | |
| CUMS + | 106.63 | 11 | 206.1 ± 2.39 | 249.0 ± 3.63 | 293.4 ± 4.14 | 340.5 ± 6.92 | 324.9 ± 6.73 | 378.5 ± 7.71**** |
| MH-1H | | | | | | | | |
| CUMS + | 106.63 | 13 | 206.1 ± 2.62 | 251.0 ± 2.99 | 291.8 ± 3.56 | 341.6 ± 6.32 | 319.1 ± 6.37 | 358.3 ± 11.34**** |
| MH-2L | | | | | | | | |
| CUMS + | 213.26 | 12 | 204.6 ± 3.53 | 245.0 ± 4.24 | 280.9 ± 4.34 | 322.4 ± 5.91 | 312.1 ± 5.91 | 351.8 ± 6.59**** |
| MH-2H | | | | | | | | |

After 5 weeks of chronic unpredictable mild stress, the body masses of the model group and the model treatment group were significantly lower than that of the normal control group, and the body mass of the model treatment group was not significantly different from that the model group, suggesting that long-term chronic mild unpredictable stress can interfere with the physiological metabolism of rats and cause body weight loss, and the intervention of positive drugs and test drugs did not improve this symptom significantly.

### 2.2 Sucrose Preference Experiment

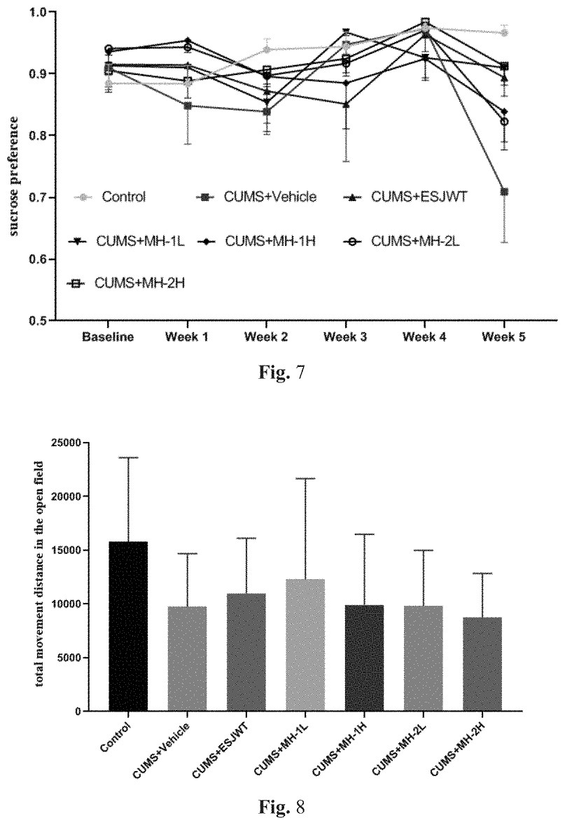

The results are shown in Table 7 and FIG. 7.

TABLE 7
The effect of the test drugs on the sucrose preference coefficient of model rats (x ± SE)

| | Dose | Number of | | | | | | |
|---|---|---|---|---|---|---|---|---|
| Group | (mg/kg) | animals | Baseline | Week 1 | Week 2 | Week 3 | Week 4 | Week 5 |
| Control | — | 14 | 0.88 ± 0.031 | 0.88 ± 0.029 | 0.94 ± 0.018 | 0.94 ± 0.024 | 0.97 ± 0.011 | 0.97 ± 0.012 |
| CUMS + | — | 12 | 0.91 ± 0.035 | 0.85 ± 0.063 | 0.84 ± 0.037 | 0.95 ± 0.015 | 0.97 ± 0.010 | 0.71 ± 0.082**** |
| Vehicle | | | | | | | | |
| CUMS + | 117.60 | 12 | 0.92 ± 0.017 | 0.91 ± 0.031 | 0.87 ± 0.052 | 0.85 ± 0.093 | 0.96 ± 0.027 | 0.89 ± 0.030## |
| ESJWT | | | | | | | | |
| CUMS + | 53.31 | 13 | 0.91 ± 0.034 | 0.91 ± 0.024 | 0.85 ± 0.047 | 0.97 ± 0.006 | 0.93 ± 0.037 | 0.91 ± 0.021### |
| MH-1L | | | | | | | | |
| CUMS + | 106.63 | 11 | 0.94 ± 0.023 | 0.95 ± 0.012 | 0.90 ± 0.017 | 0.89 ± 0.074 | 0.93 ± 0.031 | 0.84 ± 0.049 |
| MH-1H | | | | | | | | |
| CUMS + | 106.63 | 13 | 0.94 ± 0.011 | 0.94 ± 0.009 | 0.90 ± 0.025 | 0.92 ± 0.020 | 0.97 ± 0.010 | 0.82 ± 0.046 |
| MH-2L | | | | | | | | |
| CUMS + | 213.26 | 12 | 0.91 ± 0.035 | 0.89 ± 0.028 | 0.91 ± 0.024 | 0.93 ± 0.023 | 0.98 ± 0.004 | 0.91 ± 0.031### |
| MH-2H | | | | | | | | |

After 5 weeks of chronic unpredictable mild stress, the sucrose preference coefficient of the model group was significantly lower than that of the normal control group; after drug intervention, the treatment groups of Hypericum perforatum tablets, MH-1 low-dose and MH-2 high-dose could significantly improve the reduction of sucrose preference in the model group and restore it to the normal control level, suggesting that long-term chronic mild unpredictable stress can cause “anhedonia” in rats, and Hypericum perforatum tablets, MH-1 low dose and MH-2 high dose can improve this symptom in model rats.

### 2.3 Open Field Test

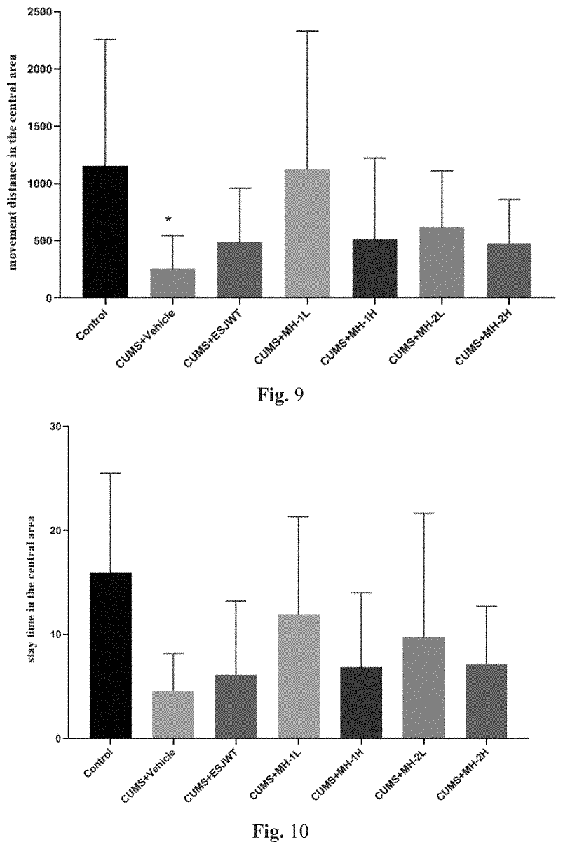

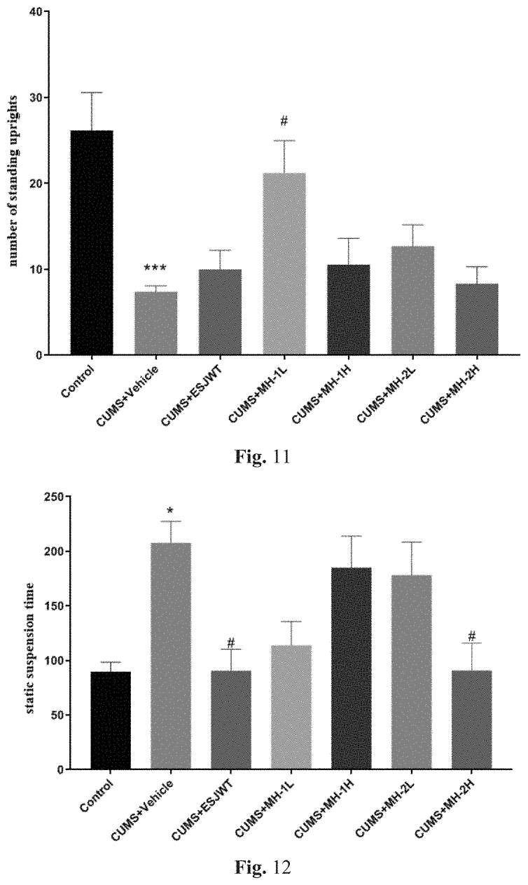

The results are shown in 8-11 and FIGS. 8-11.

TABLE 8
The effect of the test drugs on the total movement
distance in the open field of model rats (x ± SE)

| | | Number of | total movement distance |
|---|---|---|---|
| Group | Dose (mg/kg) | animals | in the open field |
| Control | — | 14 | 15803.5 ± 2082.34 |
| CUMS + Vehicle | — | 12 | 9729.3 ± 1493.48 |
| CUMS + ESJWT | 117.60 | 12 | 10995.1 ± 1541.50 |
| CUMS + MH-1L | 53.31 | 13 | 12301.00 ± 2597.14 |
| CUMS + MH-1H | 106.63 | 12 | 9918.7 ± 2073.55 |
| CUMS + MH-2L | 106.63 | 13 | 9808.9 ± 1492.40 |
| CUMS + MH-2H | 213.26 | 12 | 8759.2 ± 1226.65 |

TABLE 9
The effect of the test drugs on the movement distance
in the central area of model rats (x ± SE)

| | | Number of | Movement distance |
|---|---|---|---|
| Group | Dose (mg/kg) | animals | in the central area |
| Control | — | 14 | 1155.3 ± 306.63 |
| CUMS + Vehicle | — | 12 | 254.1 ± 97.60* |
| CUMS + ESJWT | 117.60 | 12 | 486.8 ± 149.48 |
| CUMS + MH-1L | 53.31 | 13 | 1125.9 ± 402.06 |
| CUMS + MH-1H | 106.63 | 12 | 512.9 ± 224.81 |
| CUMS + MH-2L | 106.63 | 13 | 618.9 ± 148.85 |
| CUMS + MH-2H | 213.26 | 12 | 478.4 ± 127.21 |

TABLE 10
The effect of the test drugs on the stay time
in the central area of model rats (x ± SE)

| | | Number of | Stay time in |
|---|---|---|---|
| Group | Dose (mg/kg) | animals | the central area |
| Control | — | 14 | 15.97 ± 2.883 |
| CUMS + Vehicle | — | 12 | 4.56 ± 1.140** |
| CUMS + ESJWT | 117.60 | 12 | 6.19 ± 2.227 |
| CUMS + MH-1L | 53.31 | 123 | 11.90 ± 3.340 |
| CUMS + MH-1H | 106.63 | 12 | 6.89 ± 2.521 |
| CUMS + MH-2L | 106.63 | 13 | 9.74 ± 3.593 |
| CUMS + MH-2H | 213.26 | 12 | 7.18 ± 1.966 |

TABLE 11
The effect of tested drugs on the number of
standing uprights of model rats (x ± SE)

| | | Number of | Number of |
|---|---|---|---|
| Group | Dose (mg/kg) | animals | standing uprights |
| Control | — | 11 | 26.18 ± 4.398 |
| CUMS + Vehicle | — | 10 | 7.40 ± 0.686*** |
| CUMS + ESJWT | 117.60 | 11 | 10.00 ± 2.228 |
| CUMS + MH-1L | 53.31 | 9 | 21.22 ± 3.770# |
| CUMS + MH-1H | 106.63 | 9 | 10.56 ± 3.060 |
| CUMS + MH-2L | 106.63 | 10 | 12.70 ± 2.486 |
| CUMS + MH-2H | 213.26 | 12 | 8.333 ± 1.959 |

After 5 weeks of chronic unpredictable mild stress, there was no significant difference in the total movement distance in the open field between the groups, suggesting that CUMS stress and drug intervention did not have a significant effect on the exercise and exploratory behavior of rats. After 5 weeks of chronic unpredictable mild stress, the movement distance and stay time in the central area of the model group were significantly lower than that in the normal control group, and those parameters were increased after drug intervention, but the difference was not significant. It showed that rats had enhanced anxiety-like behavior after CUMS stress, and drug intervention did not significantly improve this symptom. After 5 weeks of chronic unpredictable mild stress, the number of standing uprights in the model group was significantly lower than that of the normal control group; after drug intervention, the MH-1 low-dose treatment group could significantly improve the reduction in the number of standing uprights in the model group and restored it to the normal control level. It shows that long-term chronic mild unpredictable stress can reduce the exploratory activities of rats and decrease their interest in the external environment. Administration of low dose of MH-1 can improve this symptom in model rats.

### 2.4 Forced Swimming Test

The results are shown in Table 12 and FIG. 12.

TABLE 12
The effect of the test drugs on the static
suspension time of model rats (x ± SE)

| | | Number of | Static suspension |
|---|---|---|---|
| Group | Dose (mg/kg) | animals | time (sec) |
| Control | — | 11 | 89.8 ± 8.73 |
| CUMS + Vehicle | — | 8 | 207.8 ± 19.62* |
| CUMS + ESJWT | 117.60 | 10 | 90.7 ± 19.62# |
| CUMS + MH-1L | 53.31 | 10 | 114.0 ± 21.77 |
| CUMS + MH-1H | 106.63 | 11 | 185.1 ± 28.81 |
| CUMS + MH-2L | 106.63 | 10 | 178.2 ± 30.33 |
| CUMS + MH-2H | 213.26 | 11 | 91.0 ± 24.98# |

After 5 weeks of chronic unpredictable mild stress, the static suspension time of the model group was significantly longer than that of the normal control group; after drug intervention, the Hypericum perforatum tablets and the MH-2 high-dose treatment group could significantly improve the increased static suspension time of the model group, and restored it to the normal control level. It shows that rats with long-term chronic mild unpredictable stress have typical “behavioral despair” performance, and the Hypericum perforatum tablets and high-dose MH-2 administration can improve this symptom in model rats.

The above are only the preferred embodiments of the present disclosure. It should be pointed out that several improvements and modifications can be made by those skilled in the art without departing from the principle of the present disclosure, and such improvements and modifications should be regarded as within the protection scope of the present disclosure.

1. A traditional Chinese medicine extract composition with function of regulating depressive emotion, consisting of the following traditional Chinese medicine extracts in parts by weight: 40 to 80 parts of Hypericum perforatum extract, 10 to 40 parts of Acanthopanax senticosus extract, and 2 to 20 parts of Tree peony root bark extract.

2. The traditional Chinese medicine extract composition with function of regulating depressive emotion according to claim 1, consisting of the following traditional Chinese medicine extracts in parts by weight: 65 to 80 parts of Hypericum perforatum extract, 10 to 35 parts of Acanthopanax senticosus extract, and 2 to 10 parts of tree peony root bark extract.

3. The traditional Chinese medicine extract composition with function of regulating depressive emotion according to claim 1, consisting of the following traditional Chinese medicine extracts in parts by weight: 64 to 77 parts of Hypericum perforatum extract, 13.3 to 25 parts of Acanthopanax senticosus extract, and 2.7 to 10 parts of tree peony root bark extract.

4. The traditional Chinese medicine extract composition with function of regulating depressive emotion according to claim 1, consisting of the following traditional Chinese medicine extracts in parts by weight: 77 parts of Hypericum perforatum extract, 20 parts of Acanthopanax senticosus extract, and 3 parts of tree peony root bark extract.

5. A method of preparing the traditional Chinese medicine extract composition with function of regulating depressive emotion according to claim 1, comprising the following steps:
(1) adding 10 to 12 times weight of 60 vt % to 80 vt % ethanol aqueous solution containing 0.05% to 2% sodium hydroxide to Hypericum perforatum, extracting 2 times under heating and refluxing, 1.5 to 2.0 hours each time, combining the two filtrates and filtering, concentrating the filtrate into an extractum with a relative density of 1.11 to 1.13 at 72° C., and performing spray-drying to obtain the Hypericum perforatum extract;
(2) adding 8 to 10 times of weight of 0 vt % to 80 vt % ethanol aqueous solution to pieces of Acanthopanax senticosus, extracting 3 times under heating and refluxing, 1.0 to 2.5 hours each time, combining the three filtrates and filtering, concentrating the filtrate into an extractum with a relative density of 1.11 to 1.13 at 72° C., and performing spray-drying to obtain the Acanthopanax senticosus extract;
(3) adding 10 to 14 times weight of water to Tree peony root bark, heating and recovering 8 to 10 times amount of distillates, refrigerating for 20 to 24 hours, allowing crystallization and filtrating, and performing low-temperature drying to obtain the tree peony root bark extract; and
(4) mixing the Hypericum perforatum extract, the Acanthopanax senticosus extract and the tree peony root bark extract according to a combination ratio.

6. The method according to claim 5, wherein the content of hyperoside in the Hypericum perforatum extract is 0.1% to 3%; the content of eleutheroside in the Acanthopanax senticosus extract is 0.3% to 5%; and the content of paeonol in the tree peony root bark extract is 80% to 99%.

7. The method according to claim 5, comprising the following steps:
(1) adding 10 times weight of 80 vt % ethanol aqueous solution containing 0.1% sodium hydroxide to Hypericum perforatum, extracting 2 times under heating and refluxing, 1.5 hours each time, combining the two filtrates and filtering, concentrating the filtrate into an extractum with a relative density of 1.12 at 72° C., and performing spray-drying to obtain the Hypericum perforatum extract;
(2) adding 10 times of weight of aqueous solution to pieces of Acanthopanax senticosus, extracting 3 times under heating and refluxing, 1.0 hour each time, combining the three filtrates and filtering, concentrating the filtrate into an extractum with a relative density of 1.12 at 72° C., and performing spray-drying to obtain the Acanthopanax senticosus extract;
(3) adding 14 times weight of water to Tree peony root bark, heating and recovering 9 times amount of distillates, refrigerating for 24 hours, allowing crystallization and filtrating, and low-temperature drying to obtain the tree peony root bark extract; and
(4) mixing the Hypericum perforatum extract, the Acanthopanax senticosus extract and the Tree peony root bark extract according to combination ratio.

8. A traditional Chinese medicine preparation, comprising the traditional Chinese medicine extract composition with function of regulating depressive emotion according to claim 1 and a pharmaceutically acceptable excipient.

9. The traditional Chinese medicine preparation according to claim 8, wherein the traditional Chinese medicine preparation has a dosage form of capsule or tablet.

10. The traditional Chinese medicine preparation according to claim 8, wherein the content of the traditional Chinese medicine extract composition with function of regulating depressive emotion is not less than 70% by weight in the traditional Chinese medicine preparation.
